(19) United States
(12) Patent Application Publication
(10) Pub. No.: US 2023/0282327 A1
(43) Pub. Date: Sep. 7, 2023

(54) METHOD FOR GENERATING AN INDICATOR OF COMPLIANCE WITH A DRUG TREATMENT, SYSTEM

(71) Applicant: DRAGO, CHARTRES (FR)

(72) Inventors: Julien COTTET, CHARTRES (FR); Mickaël POULIQUEN, LIFFRE (FR); Eric FROMENTIN, LILLE (FR); Hoai-Bich TRINH, OLIVET (FR); Edouard SEVE, OLIVET (FR)

(21) Appl. No.: 18/006,111

(22) PCT Filed: Jul. 19, 2021
(86) PCT No.: PCT/EP2021/070188

§ 371 Date: Jan. 19, 2023

(30) Foreign Application Priority Data
Jul. 20, 2020 (FR) FR2007580

(51) Int. Cl. G16H 20/10

(52) U.S. Cl. CPC G16H 20/10

(57) ABSTRACT

A method for generating a graphical indicator of compliance with a drug treatment includes identification of a user, activation of a first program resulting in the activation of a timer and generation of a time marker; generation of a first score counting the generation of the generated time marker and a set of time markers previously generated; activation of a second program collecting a set of user interactions on a digital interface to generate a second score; activation of accessibility to a set of first areas when the first score is greater than a first threshold for the number of days; activation of accessibility to a set of second areas when the second score is greater than a value, and generation of a numerical avatar defining an indicator of compliance with the treatment.

### FIELD OF THE INVENTION

The invention relates to the field of methods and devices for verifying, checking, and monitoring compliance with a treatment by an individual. The field of the invention relates more particularly to methods for generating indicators aiming to measure and quantify compliance with a treatment from an electronic device.

### PRIOR ART

Solutions that make it possible to check that a treatment is correctly followed exist. For example, in order to check the daily intake of a pill, pill boxes or other devices exist that can be used to determine the status of monitoring a treatment by a patient. For some drugs, there are also radio markers that make it possible to retrieve information on taking a drug and thus check compliance with a treatment, such as the case for example of the drug Abilitify MyCite©. This solution is suitable for one type of drug, notably those that dissolve in the stomach. This solution is not suitable for all treatments, notably those involving dissolution of the drug in the mouth or its restitution after storage for a duration of few minutes.

However, when a treatment has to be taken daily over a long period, for example a period exceeding one year, and when the directions for use requires mobilization of an individual for several minutes each day, it is known that compliance may be low. This is notably the case for a treatment aimed at desensitizing a person to a respiratory allergen. This treatment requires an individual to take a daily treatment for more than three years. In this treatment, the individual must keep a solution in the mouth for several minutes. This type of treatment results in discouragement for the individual over time, and the latter gradually loses the requirement of a daily appointment that he considers as a burdensome constraint.

To this end, there are methods for coaching or creating applications to promote the mobilization of an individual on a daily basis. However, these applications are sometimes poorly suited to certain treatments and most often rely on questioning of the individual to determine the most suitable answers to provide them as described in the application WO2019/099557. This solution is not suitable for administration of a drug which is daily and therefore repetitive.

There is a need to define a solution that can be used to define an indicator of compliance with a treatment and that can furthermore be a solution that promotes an increase in said compliance with a treatment.

### SUMMARY OF THE INVENTION

According to a first aspect, the invention relates to a computer implemented method for generating a graphical indicator of compliance with a drug treatment comprising the following steps:
Identification of a user by a remote user rights management server, said identification parameters being entered from a first user interface of a first electronic terminal,
Activation of a first computer program on a current date leading to the activation of a chronometer on a “launch date”, and generation of a time marker for taking a daily dose of drug treatment, said first program being executed on the first terminal;
Generation of a first score counting the generation of said generated time marker and a set of time markers previously generated on dates prior to the current date and counted since an initial date defined by the user, said first score being calculated from a calculator and a date data stored in the terminal;
Activation of a second computer program collecting a set of user interactions for the first predefined time on a digital interface of said electronic terminal to generate a second daily/everyday score, said second program being executed on the first terminal;
Activation of an accessibility to the first user to a library of a subset of first graphic areas when the first score is greater than at least one predefined first threshold number of days;
Activation of an accessibility to the first user to a library of a subset of second graphic areas when an evolution index is greater than a first predefined value, said evolution index comprising at least one combination of a second daily score and of a first score, the second graphic areas and the first graphic areas defining predefined numerical graphic elements saved in a memory of a remote server, each first graphic area combined with a selection of a subset of second graphic areas defining a numerical avatar,
Automatic generation of a numerical avatar defining an indicator of compliance with the treatment comprising a first graphic area and a selection of second graphic areas each time the first computer program is relaunched as a function of the evolution index.

One advantage of the invention is to generate a marker of compliance with the treatment of a drug registered over a long period, such as several years, and for which a daily intake is carried out for several minutes.

According to one embodiment, the first program comprises a function to define the initial date corresponding to the start of the treatment, all of the dates between the initial date and the current date are days. Each time stamp corresponds to the count of one day.

According to one embodiment, the value of the second daily/everyday score is less than a maximum value, said maximum value being defined by all the interactions generated in the first predefined duration or by a predefined set score.

One advantage is that the number of interactions considered when calculating the score is limited to the time window during which the treatment must be retained. Thus, a maximum value is defined to return an actual compliance status.

According to one embodiment, the first marker at a current date is generated when the chronometer duration reaches a first predefined duration. One advantage is to ensure that the treatment is actually taken for the duration of the daily treatment.

According to one embodiment, the evolution index comprises:
A combination of a daily gain of the first score and the second daily score and/or;
A combination of the first score and the second daily score and/or;
A combination of the first score and a sum of daily/everyday second scores calculated over a date interval;

One advantage is to make it possible to generate a graphical indicator making it possible to evolve according to a chronology segmented by steps. Thus, this definition of steps allows a user to obtain an evolving graphic control, the evolution of which reflects steps or milestones of his treatment.

According to one embodiment, the first program comprises a function for automatically activating the second program.

According to one embodiment, interactions generated on a given date beyond the predefined duration are not counted in the second score.

According to one embodiment, the predefined duration is less than 3 minutes and corresponds substantially to the duration of administration of a drug to be maintained in the mouth.

According to one embodiment, a plurality of predefined durations makes it possible to count time markers of different values.

According to one embodiment, a set of thresholds for the number of days is predefined so that a predefined number of first graphic areas can be generated during the duration of the treatment.

According to one embodiment, the reaching of a value of the first score and/or the evolution index results in the accessibility to a third program from the first program, the third program making it possible to offer another mode of interaction to collect points of the second score.

According to another aspect, the invention relates to a computer program product downloadable from a communication network and/or stored on a data carrier readable by a computer and/or executable by a computer, said computer program product comprising computer program code instructions for implementing the method of the invention.

According to another aspect, the invention relates to a computer readable data storage medium on which is stored a computer program comprising program code instructions for implementing the method of the invention.

### BRIEF DESCRIPTION OF THE FIGURES

Other characteristics and advantages of the invention will be become clearer on reading the following detailed description, with reference to the appended figures, that illustrate:

FIG. 1: an exemplary embodiment of implementing a method of the invention;

FIG. 2: an example of a system architecture for implementing the method of the invention;

FIG. 3: an example of interface of the second program of the invention making it possible to collect interactions with the user;

FIG. 4: a first example of a graphical indicator of a patient's compliance with a treatment presented in the form of an avatar.

FIG. 5: a second example of a graphical indicator of a patient's compliance with a treatment presented in the form of an avatar.

### DETAILED DESCRIPTION

The invention relates to a method for generating a graphical indicator in the form of an avatar that can evolve over time. The avatar evolves as a function, on the one hand, of the regular intake, notably daily, of a treatment and, on the other hand, data allowing concrete qualification of whether an individual has followed the directions for use for a certain period of time during the intake of a drug. The direction for use is, for example, to keep a drug under the tongue or in the mouth and then swallow it or spit it out. Other directions of use could be envisaged within the scope of the invention: taking a drug until it melts or successively taking a plurality of drugs or application on the skin of a product and massage, etc.

The avatar AV thus appears as a numerical control or indicator of compliance with a treatment insofar as its evolution considers, on the one hand, a datalinked to the maintenance of a drug for a certain time in the mouth and, on the other hand, the regularity of administration.

The invention makes it possible to evolve the avatar AV according to user interactions thanks to a program PRG₂ that makes it possible to maintain the effort and attention of the user during the time of the drug intake which lasts several minutes. Thus, considering the interactions of the program PRG₂ allows the avatar AV to evolve and thus contributes not only to measuring compliance, but also to increasing it.

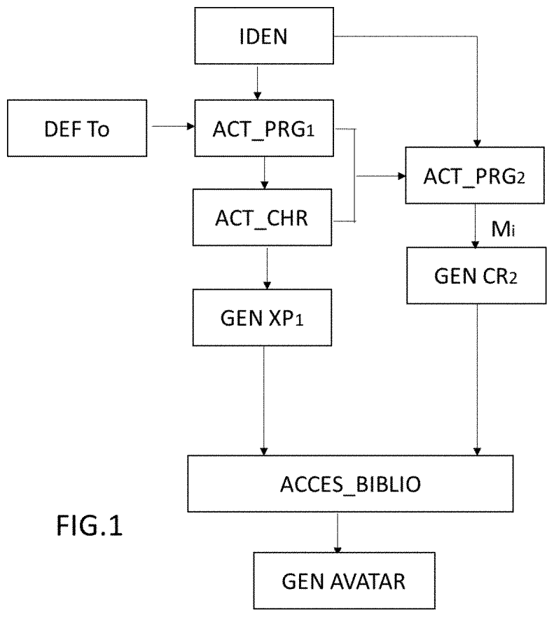

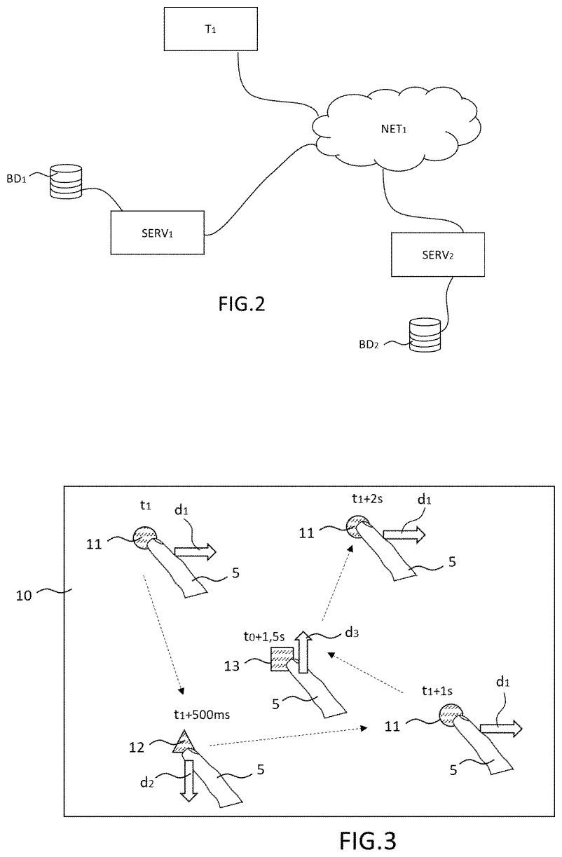

FIG. 1 shows an exemplary implementation of the method of the invention through different steps. The system of the invention is described through the elements of FIG. 2. The following description gives an exemplary embodiment of the implementation of a method of the invention and a system of the invention.

A first step noted IDEN makes it possible to identify a user U₁ of an electronic terminal T₁. Identification is performed from a computer program, otherwise called application, and a user interface of an electronic terminal T₁ in which an individual U₁ can define an identifier and a password. In order to identify a user U₁, an access rights management server SERV₁ can be implemented within a data network NET₁ such as an Internet. The invention may be implemented with any method of authentication or identification of a user U₁ with a rights management server. The electronic terminal T₁ may be, for example, a Smartphone, a tablet, a computer, or an electronic equipment with a communication interface for connection to the data network.

The identification makes it possible to ensure that the method is correctly executed for a same user U₁ at each daily drug administration and to recover, if necessary, from the network, a user profile or preferences or graphic elements necessary to generate an avatar AV.

The method represents a second step of activation ACT_PRG₁ of a first program PRG₁. This step may be performed successively to the identification step IDEN. According to an example, the identification IDEN is ensured by the first program PRG₁. This step may, for example, be automatically initiated when a mobile application installed on a Smartphone is launched. At the first launch of the program PRG₁, the user U₁ is invited to initiate the date of the start of the treatment T₀, which will last several years. According to an example, this is the current date.

According to one embodiment, the activation of the first program PRG₁ results in the start of a time counter, i.e., a timer CHR. According to another example, a command is engaged by the user U₁ in order to start the timer CHR. For example, a command generated by pressing a button or a first touch command makes it possible to start the timer CHR. This last possibility makes it possible to start the timer CHR when the user U₁ of the terminal T₁ has placed the drug in his mouth.

This function of timer CHR may be performed from a clock and a calculator of the terminal T₁. When the timer CHR is launched, a second program PRG₂ starts, this step is noted ACT_PRG₂. This second program PRG₂ may be launched automatically when starting the timer CHR or manually engaged by the user U₁.

According to another embodiment, the second program PRG₂ may be launched directly after the authentication of the user U₁. In the latter case, it may be launched at the same time as the first program PRG₁ or at the same time as the start of the timer CH₁ or at the same time as the first command. According to an exemplary embodiment, the first program PRG₁ allows access to a set of programs PRGᵢ, so the user U₁ may thus choose the computer program he wants to launch or the one suggested to him, such as the program PRG₂.

The second program PRG₂ is configured to generate a user interface that can collect interactions of the user U₁. These interactions are collected and counted using the resources of the terminal T₁, such as its calculation means and detection means, for example, of a touch screen. The interactions may comprise interactions with the touch screen, actions on an actuator such as an electronic or mechanical button, or voice commands acquired by a microphone of the electronic terminal T₁. The interactions may also comprise, in some embodiments, measurements of a sensor, such as an optical sensor of a camera.

FIG. 3 shows an example of a user interface 10 generated by the second program GWP₂. In this example, patterns 11, 12, 13 are generated over time at different moments t₁, t₁+500 ms, t₁+1.5 s t₁+2 s each pattern being displayed for a short instant, for example within a range of the order of 500 ms to 3 s. The user U₁ must then perform an action on the interface, such as swiping his finger 5 on the interface 10 in a given direction d₁, d₂, d₃ according to the pattern appearing. The direction of movement of the finger 5 must then conform to the appearance of a given pattern, such as one of the patterns 11, 12, 13 to make it disappear. According to one example, the following combinations can be made: {11, d₁}, {12, d₂}, {13, d₃}.

This example makes it possible to illustrate a way of generating interactions of a user U₁ for a duration at least comprised within the time interval required for the daily administration of the medication. The interactions make it possible to generate a second daily score CR₂. This step is noted GEN_CR₂ in FIG. 1. According to one example, the second daily score CR₂ may be a score counting the touches and/or movements of at least one finger on the screen of the terminal T₁. In the case of movements, the movements may be counted according to their directions in accordance with events occurring. According to one example, the second daily intake CR₂ considers the total number of interactions over a given period of time. According to one example, the second daily score CR₂ takes a data relative to the speed of execution of the movement and the sequence of the different movements. According to one example, the speed at which the patterns appear may increase as a function of the response of the user U₁ and thus as a function of the speed at which the movements and their sequences are executed. According to one example, the rate of appearance of the patterns 11, 12, 13 may increase as a function of the current date with respect to the initial date T₀ of the first program PRG₁ and/or the first score XP₁.

According to one embodiment, the second program PRG₂ comprises different levels that can be accessed either after obtaining a second sufficient daily score CR₂ during a daily session, or after obtaining an evolution index corresponding to a plurality of daily scores CR₂ accumulated over a period of several daily sessions also considering the first score XP₁.

According to another example, the level may also change by considering the first score XP₁ in combination with at least one second daily score CR₂.

Other examples of user interface 10 may be implemented in the invention. The patterns and the interactions may be more complex and be generated within a graphical environment that can evolve. According to an example, this can be a function collecting interactions within a game, for example the pattern may be a character evolving in a graphic environment.

According to one embodiment, based on a value of an evolution index calculated from the first score XP₁ and/or the second daily score CR₂ and/or a score calculated from several daily scores CR₂, a third program PRG₃ may be accessible to a user U₁. The third program PRG₃ provides another mode of interaction in order to collect points to generate a new second daily score CR₂. The evolution index calculated from the first score XP₁ and at least one new second daily score CR₂ makes it possible to generate an avatar AV having new graphic areas BASᵢ, MEBᵢ.

A first effect of considering these interactions is to measure the fact that the user U1 is actually in the course of monitoring his treatment by accomplishing his taking of the medication. Indeed, by launching the first program PRG₁, the timer CHR is engaged and by launching the second program PRG₂, the user U₁ is invited to interact with the user interface of the terminal T₁ while keeping a drug in his mouth for several minutes.

A second effect of considering these interactions is that the evolution index will allow the graphical representation of the avatar AV to evolve to generate a graphical indicator of compliance with the treatment and, more generally, monitoring of the treatment of the individual U1. Indeed, as the treatment continues and the user U₁ advances his first score XP₁, he can successively or alternately progress to change level within a same program, such as the second program PRG₂, or trigger access to a new program, such as the third program PRG₃, which has the effect of allowing his avatar to evolve.

When the duration of the timer CHR exceeds a certain predefined duration, a time marker Mᵢ is generated validating that the user U₁ has maintained his medication in the mouth for a minimum duration. The time marker Mi is associated with a date Tᵢ. Thus, the first score XP₁ may comprise a calculation of the number of time markers Mᵢ since the initial date T₀ of the start of treatment and therefore the initial date T₀ of launch of the first program PRG₁. Thus, on the 50th day of treatment, if the user U₁ has taken his daily treatment for the duration indicated and as many time markers Mᵢ have been generated, then the first score XP₁ may be calculated as follows: XP₁= [symbol] (Mᵢ)_{iε[1,50]}. If one marker Mi is equal to one unit. The first score XP₁=50.

This first score XP₁ corresponds to a user experience and gives an indication since the start of the treatment. If the user has only taken his treatment 40 times since the initial date T₀ and we are at the 50^{th} day. The experience of the user U₁ allows a lower score XP₁ to be accumulated than in the previous case, i.e., a score of 40 calculated under the same conditions as previously with 40 time markers Mᵢ generated. Only the medication taken by the individual over the time interval necessary for taking the medication is counted.

According to other examples, the first score XP₁ may be calculated by considering other criteria in order to weight or calculate a composite score. These other criteria may be combined with the time markers Mᵢ to consider the user experience with another criterion.

As an example, the first score XP₁ may consider a criterion improving the score when the treatment has been taken for a certain number of consecutive days or even a criterion penalizing the score when the treatment has been interrupted several days in a row.

According to one embodiment, the first score XP₁ influences the calculation of the evolution index in order to consider a type of first graphic areas compatible with second graphic areas. Compatibility may correspond to area sizes, area colors or shape contours of said areas which make it possible to match a first graphic area BASᵢ with a second graphic area MEBᵢ. In the latter case, the first score XP₁ is made up of a fixed daily score and a score dependent on the second daily score CR₂.

The first score XP₁ is used to give the user U₁ access to a graphic library in which first graphic areas, also called morphological bases {BASᵢ}_{iε[1;7]}, may be chosen as a function of the experience acquired by the user U₁. This step is noted ACCES_BIBLIO in FIG. 1. Access to morphological bases {BASᵢ}_{iε[1;7]} makes it possible to measure long compliance, i.e. the capacity of an individual U₁ to continue his treatment over the long term.

According to one example, 7 morphological bases BASᵢ defining the body of an avatar AV may be accessed successively by a user U₁. The 7 morphological bases {BASᵢ}_{iε[1;7]} are associated with 7 scores XP₁, which can be reached successively by continuing treatment. In the case where the first score XP₁ corresponds to a number of days of treatment considered to have been observed, a possible segmentation is as follows: {BAS₁, 3}, {BAS₂, 90}, {BAS₃, 90}, {BAS₄, 180}, {BAS₅, 180}, {BAS₆, 180}, {BAS₆, 365}, where 3, 90, 180 and 365 are the number of days required to gain access to the next morphological database within the graphic library. This segmentation makes it possible to evolve a graphical indicator by modifying the body of the avatar AV. Thus, as a living being, the avatar AV grows, thus providing an indicator specific to a coherent evolution of the monitoring of compliance with the drug treatment by the individual U₁.

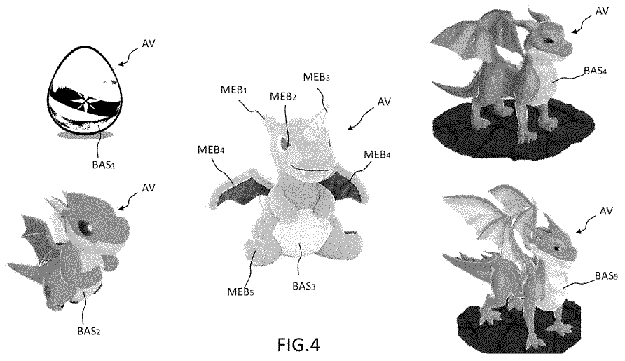

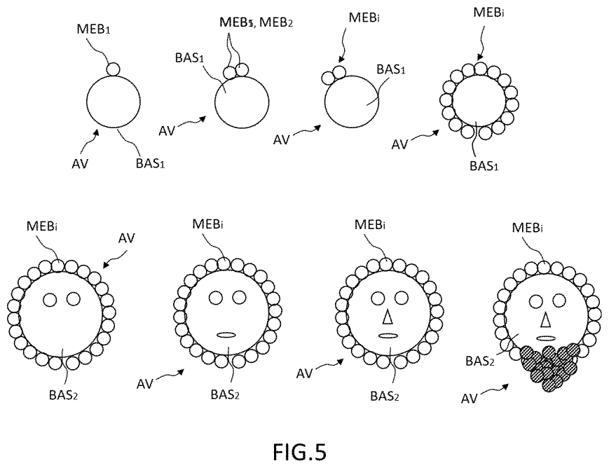

FIG. 4 shows an example of an avatar AV evolving over time as the drug treatment is observed by an individual. Five morphological bases BAS₁, BAS₂ BAS₃, BAS₄, BAS₅ of an avatar AV representing successively an egg, a little dragon and then a dragon are shown. The morphological base BAS₃ is represented with different second graphic areas, called morphological areas MEBᵢ. Other examples of avatars may be used within the scope of the invention. These may be fictitious characters, real characters, characters representing a human, or characters representing an animal, etc. The type of character chosen to represent the avatar does not matter from the point of view of the technical effect produced. The invention makes it possible to evolve an avatar AV regardless of its initial type in order to constitute an indicator of compliance with a treatment. The technical aspect of the indicator lies in the ability to take into consideration a long factor: the regularity of the treatment and a short factor: the taking of the treatment up to the end. The product indicator makes it possible to reproduce these two aspects in order to better quantify and qualify compliance with a drug treatment having important constraints for an individual.

The value of the evolution index calculated from the second score CR₂ and possibly the value of the first score XP₁ makes it possible to give the user U₁ access to a graphic library in which second graphic areas, called morphological areas {MEBᵢ}_{iε[1,7]} can be chosen. This step is also noted ACCES_BIBLIO in FIG. 1.

Access to morphological areas MEBᵢ makes it possible to quantify the assiduity of taking the treatment daily until the end of the time required for its administration. It corresponds to a daily compliance with the daily treatment.

According to one example, for each morphological base BASᵢ defining a body of an avatar AV, a set of morphological areas MEBᵢ may be associated with said morphological base BASᵢ.

Each morphological area MEBᵢ makes it possible to define an element of the graphic library which can be made accessible according to the value of the evolution index, the latter being calculated from the second score CR₂ and the first score XP₁.

Thus, a user U₁ interacting with the user interface of the second program PRG₂ for the duration of the treatment acquires a score CR₂ which allows him to access additional morphological areas MEBᵢ. One benefit is to obtain an indicator of compliance with the daily treatment. It is possible to measure to what extent the treatment is followed during an administration.

The first score XP₁ and the evolution index notably based on the second daily scores CR₂ make it possible to define two indicators translating whether a treatment is correctly taken by a user U₁ throughout the duration of the treatment and whether it is correctly taken during each administration up to the time required for administration. Thus, the avatar AV generated is a true indicator of compliance with a treatment. The avatar generation step is noted GEN_AVATAR. The avatar AV is preferably generated at the start of each launch of the program PRG₁.

According to one example, the morphological areas MEBᵢ may comprise:
limbs or parts of a person's body representing an avatar AV, such as arms, legs, or feet MEB₅, ears MEB₁ or eyes MEB₂,
morphological particularities such as horns MEB₃, wings MEB₄,
accessories such as rings, crowns, bracelets of ornaments,
coatings such as skins, tattoos, textures such as scales or clothes such as capes.
animations defined on an avatar area, for example: spit fire, jump of the avatar, a wink, etc.

One benefit is to build an avatar AV that evolves and forms an indicator of the monitoring of the treatment. The more an individual follows the treatment, the more he is encouraged to collect and enrich the avatar AV.

In the invention, it is understood that the generation of an avatar AV has an interest, because it makes it possible to quickly obtain an assessment of compliance with a treatment, notably by offering an evolving graphical representation reproducing an implicit time reference. This would be more difficult with geometric shapes that do not form part of a whole that evolves over time.

The invention may also apply to other graphical representations in so far as the chosen form enables a temporal evolution to be reproduced considering a daily evolution of the taking of a treatment.

According to one example, the shape may be colored in a first initial state with a first color and may evolve to another shape having another color throughout the treatment as the treatment is taken by the individual. However, it is understood that such a representation remains limited in the consideration of information relating to the qualification of taking treatment throughout the duration of administration, i.e. for several minutes. Indeed, in order to return this information, it is more effective to add to the initial form other elements enriching the latter to consider the fact that administration of the drug has been up to the end of the duration of administration of the drug.

The example of FIG. 5 is another example in which a morphological base BAS₁ is initiated at the start of treatment. The initial date T₀ leads to the generation of a first basic form, in this case a circle. In the case of FIG. 4, a first form BAS₁ may be an egg. As the treatment is taken daily by the individual U₁, the invention makes it possible to measure at each administration a second score CR₂ making it possible to assess whether the user has interacted with the second program GWP₂. Thus, it is possible to qualify the efficacy of taking the drug. The second daily score CR₂ or a score considering the evolution of the second daily scores CR₂, such as the evolution index, makes it possible to collect morphological areas MEBᵢ, in this case the smallest circles arranged around the morphological base BAS₁.

At the end of several days, it is understood that the avatar AV evolves with the same morphological base BAS₁ by the addition of morphological area MEBᵢ. The first threshold not yet having been crossed to evolve the morphological base BAS₁, the user U₁ obtains, thanks to the increase in the second daily score CR₂, new morphological areas MEBᵢ.

From a certain number of days when the treatment is taken, the morphological base BAS₁ changes and becomes BAS₂ which corresponds to a larger circle than that of BAS₁. The avatar AV grows as the first score XP₁ crosses thresholds. More generally, a change in the geometric shape of the morphological base allows the avatar AV to evolve.

By continuing to interact for a few minutes daily during the administration of the drug, the user U₁ acquires an evolution index allowing him to evolve the avatar AV. It is therefore possible to qualify the fact that the drug is taken to the end, because the user has interacted during the taking of the drug. Without interaction, it is not possible to collect enough points to evolve a score, such as the evolution index, calculated on the basis of second daily scores CR₂ and therefore to evolve the avatar AV with new morphological areas MEBᵢ. According to one embodiment, a succession of second daily scores CR₂ is required to evolve the avatar AV.

The evolution of the avatar AV is moreover encouraging for the user, who is incited to follow his treatment appropriately. Thus, the avatar AV is an indicator of compliance with a treatment which also has the effect of increasing compliance by encouraging the user U₁ to grow his avatar AV.

FIG. 2 shows an example of a system of the invention comprising a terminal T₁ of a user U₁ that is possibly connected to a data network NET₁. The system includes an access rights management server SERV₁ and possibly a database for storing identifiers and authentication data. In addition, the system includes a server SERV₂ for storing data specific to the programs PRG₁, PRG₂, and user profile data or interaction data of an individual U₁ such as scores or accessible elements of the graphical library. For example, the programs PRG₂, PRG₃ and any other program that can be run from the terminal T₁ may be downloaded from the server SERV₂ or from another application download server (not shown). According to one embodiment, the programs PRG₂, PRG₃, etc. are loadable on the terminal T₁ to be executed on said terminal T₁. According to another example, the programs are run on the server SERV₂, in which case the terminal T₁ maintains a wireless connection. According to another case, the second program PRG₂ is executed on the terminal T₁ and the first program PRG₁ is executed on the server SERV₂. According to a preferred mode, the programs PRG₁ and PRG₂ are executed on the terminal T₁. According to an example, only certain specific functions are performed on the remote servers SERV₂, such as access to a library of morphological elements, score calculation or the function allowing an individual to be authenticated.

The invention makes it possible to implement a function in the program PRG₁ and/or PRG₂ or any other program of the invention implementing data of individuals for sharing said data in a community of users. An interest is to encourage an individual U₁ to continue his treatment by pooling certain data such as the scores obtained or his avatar AV. When a social or community function is implemented, it makes it possible to exchange data between different individuals who have installed the program PRG₁ and the program PRG₂ and possibly other programs PRG₃. In this context, a remote memory, or the database BD₂, for example that of the server SERV₂, makes it possible to store user data in order to establish comparable. Among the functions, rankings, suggested actions, alert generations, and data exchanges may be triggered automatically in order to have the different individuals in a same community interact. The community is preferably formed within a program such as the program PRG₂ or PRG₃ or any other program accessible to the user from the program PRG₁.

1. A computer implemented method for generating a graphical indicator of compliance with a drug treatment, the computer implemented method comprising:
identifying a user by a remote user rights management server, wherein identification parameters are entered from a first user interface of a first electronic terminal,
activating a first computer program on a current date leading to an activation of a chronometer on a launch date, and generating a time marker for taking a daily dose of drug treatment, said first computer program being executed on the first electronic terminal;
generating a first score counting a generation of said generated time marker and a set of time markers previously generated at dates prior to the current date and counted since an initial date defined by the user, said first score being calculated from a calculator and a date data stored in the first electronic terminal;
activating a second computer program collecting a set of interactions of a user during the first predefined time on a digital interface of said first electronic terminal to generate a second daily score, said second computer program being executed on the first electronic terminal;
activating an accessibility to the first user to a library of a subset of first graphic areas, each defining a morphological base, when the first score is greater than at least one first predefined number of days threshold;
activating an accessibility to the first user to a library of a subset of second graphic areas each defining a morphological area, when an evolution index is greater than a first predefined value, said evolution index comprising at least one combination of a second daily score and of a first score, the second graphic areas and the first graphic areas defining predefined numerical graphic elements stored in a memory of a remote server, each first graphic area combined with a selection) of a subset of second graphic areas defining a numerical avatar, and
automatically generating a numerical avatar defining an indicator of compliance with the drug treatment comprising a first morphological base and selecting morphological areas selecting second graphic areas at each new launch of the first computer program as a function of the evolution index.

2. The computer implemented method according to claim 1, wherein the first computer program comprises a function to define the initial date corresponding to a start of the drug treatment, each date of the set of dates between the initial date and the current date is one day.

3. The computer implemented method according to claim 1, wherein a value of the second daily score is less than a maximum value, said maximum value being defined by all the interactions generated in the first predefined duration or by a predefined set score.

4. The computer implemented method according to claim 1, wherein a first marker at a current date is generated when a duration of the chronometer reaches a first predefined duration.

5. The computer implemented method according to claim 1, wherein the evolution index comprises:
a combination of a daily gain of the first score and the second daily score and/or;
a combination of the first score and the second daily score and/or;
a combination of the first score and a sum of second daily scores calculated over an interval of dates;

6. The computer implemented method according to claim 1, wherein the first computer program comprises an automatic activation function of the second computer program.

7. The computer implemented method according to claim 1, wherein interactions generated on a given date beyond a predefined duration are not counted in the second score.

8. The computer implemented method according to claim 1, wherein a set of thresholds of the number of days is predefined so as to allow the generation of a predefined number of first graphic areas during the duration of the drug treatment.

9. The computer implemented method according to claim 1, wherein reaching a value of the first score and/or the evolution index results in the accessibility to a third computer program from the first computer program, the third computer program making it possible to offer another mode of interaction to collect points of the second score.

10. (canceled)

11. A non-transitory data computer readable medium, on which is stored a computer program comprising program code instructions for implementing the method according to claim 1.
